(19) United States
(12) Patent Application Publication
(10) Pub. No.: US 2008/0098626 A1
(43) Pub. Date: May 1, 2008

(54) Healing Shoe and Decorative Strap Therefor

(76) Inventor: Willie Wright, Savannah, GA (US)

Correspondence Address: NIXON & VANDERHYE, PC, 901 NORTH GLEBE ROAD, 11TH FLOOR, ARLINGTON, VA, 22203, US

(21) Appl. No.: 11/871,310

(22) Filed: Oct. 12, 2007

Related U.S. Application Data
(60) Provisional application No. 60/855,291, filed on Oct. 30, 2006.

(51) Int. Cl. A43B 23/24; A43C 11/00; A61F 5/01

(52) U.S. Cl. 36/136; 36/110; 36/50.1

(57) ABSTRACT

A healing shoe includes a shoe sole, a shoe upper attached to the shoe sole, and at least one portion that folds over a wearer's foot. A strap is selectively attachable to the at least one portion and secures the shoe on the wearer's foot. At least one decorative attachment is secured to the strap.

### CROSS-REFERENCES TO RELATED APPLICATIONS

This application claims the benefit of U.S. Provisional Patent Application Ser. No. 60/855,291, filed Oct. 30, 2006, the entire content of which is herein incorporated by reference.

### STATEMENT REGARDING FEDERALLY SPONSORED RESEARCH OR DEVELOPMENT

(NOT APPLICABLE)

### BACKGROUND

The invention relates to a decorative walking boot or healing shoe and, more particularly, to a decorative strap for such a shoe.

After suffering a foot injury or after surgery or other trauma, patients are often required to use a walking boot or healing shoe that properly supports the foot while enabling the affected area to heal. Examples of such shoes are disclosed in, for example, U.S. Pat. No. 4,726,127, U.S. Pat. No. 5,483,757, and U.S. Pat. No. D534,712, the disclosures of which are hereby incorporated by reference.

Typically, such shoes are unattractive or awkward. It would thus be desirable to provide such a walking boot or healing shoe with a decorative feature to enhance the appearance of the shoe.

### SUMMARY

A decorative walking boot or healing shoe is provided with one or more decorative attachments applied to a shoe strap and/or other exterior surfaces of the shoe. The resulting shoe is attractive and colorful.

In an exemplary embodiment, a healing shoe includes a shoe sole, a shoe upper attached to the shoe sole and including at least one portion that folds over a wearer's foot, and at least one strap selectively attachable to the at least one portion to secure the shoe on the wearer's foot. At least one decorative attachment is secured to the strap. In one arrangement, the shoe includes two portions that fold over the wearer's foot, and the at least one strap is selectively attachable to the two portions to secure the shoe on the wearer's foot. Preferably, the at least one strap is completely removable. In this context, the shoe may include a plurality of interchangeable straps respectively including a different decorative attachment.

The decorative attachment may comprise one of more of fabric, paint, beads, costume jewelry and the like.

In another exemplary embodiment, a healing shoe includes a shoe sole, a shoe upper attached to the shoe sole and including at least one portion that folds over a wearer's foot, and a plurality of interchangeable straps selectively attachable to and detachable from the at least one portion. At least one decorative attachment is secured to each of the straps, the decorative attachment being different on each of the straps such that each strap comprises a unique appearance.

### BRIEF DESCRIPTION OF THE DRAWINGS

These and other aspects and advantages will be described in detail with reference to the accompanying drawings, in which:

FIG. 1 is a perspective view of the healing shoe including one or more decorative attachments on the securing strap; and

FIG. 2 shows a plurality of exemplary decorative straps.

### DETAILED DESCRIPTION OF PREFERRED EMBODIMENTS

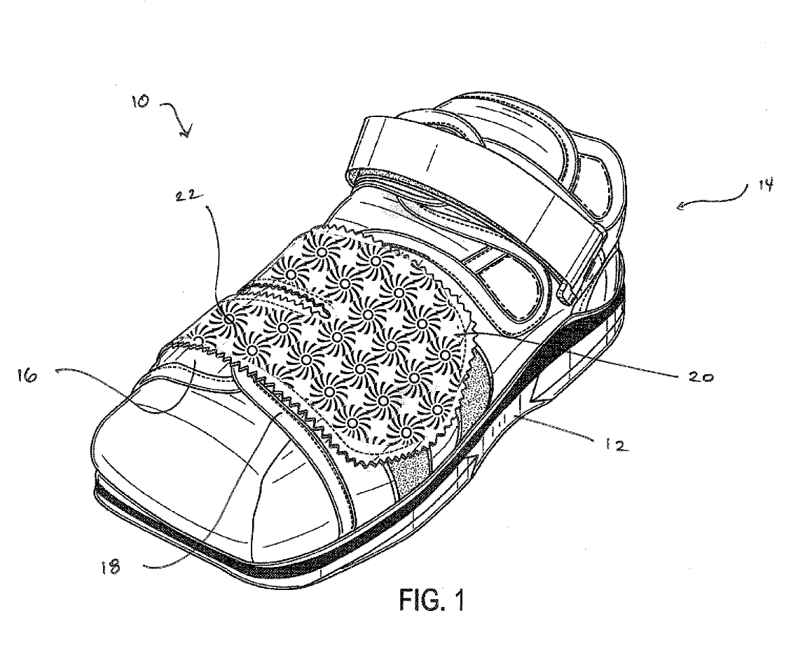

With reference to FIG. 1, a walking boot or healing shoe 10 includes a shoe sole 12 and a shoe upper 14. In a sandal style shoe as shown, the upper 14 is formed with a first overlap piece 16 that is folded over the wearer's foot, and preferably a second overlap piece 18 that is folded over the first overlap piece 16. A strap 20 including a suitable fastener, such as a hook and loop fastener or the like, is fixed over the first and second overlap pieces 16, 18 to selectively secure the second overlap piece 18 to the first overlap piece 16, and thereby secure the wearer's foot on the shoe sole. In a preferred construction, the strap 20 is completely removable from the shoe 10.

At least one decorative attachment 22 is provided on the strap 20. The decorative attachment 22 may be comprised of fabric, paint, beads, costume jewelry, and/or any other decorative item as well as any combination thereof. The decorative attachment shown in FIG. 1 is merely exemplary.

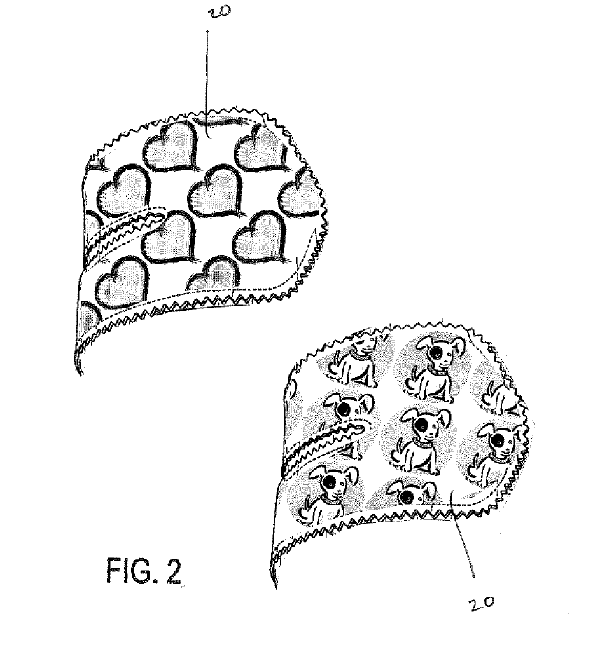

Since the strap 20 is preferably completely removable from the shoe 10, the shoe 10 may be provided with a plurality of straps 20 each including a different decorative attachment 22 as shown in FIG. 2. Since the straps 20 are interchangeable, the shoe 10 can be worn with multiple different looks.

The decorative attachment(s) 22 may additionally be placed on any exterior surface of the sole 12 or upper 14. Of course, any arrangement of fabrics, jewels and colors may be used, and the invention is not meant to be limited to the illustrated examples.

Although the strap 20 has been described in the context of its use with an exemplary healing shoe, the strap 20 may come in different shapes and sizes for use with any type of shoe, whether serving to secure the shoe on the wearer's foot or not. That is, existing shoes may be fitted with attachment structure, such as one part of a hook and loop fastener, cooperable with corresponding attachment structure, such as the other part of a hook and loop fastener so that the strap 20 and decorative attachment 22 can be affixed to any type of shoe.

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and scope of the appended claims.

1. A healing shoe comprising:
a shoe sole;
a shoe upper attached to the shoe sole and including at least one portion that folds over a wearer's foot;
at least one strap selectively attachable to the at least one portion to secure the shoe on the wearer's foot; and
at least one decorative attachment secured to the strap.

2. A healing shoe according to claim 1, comprising two portions that fold over the wearer's foot, wherein the at least one strap is selectively attachable to the two portions to secure the shoe on the wearer's foot.

3. A healing shoe according to claim 1, wherein the at least one strap is completely removable.

4. A healing shoe according to claim 3, comprising a plurality of straps respectively including a different decorative attachment, wherein the straps are interchangeable.

5. A healing shoe according to claim 1, wherein the decorative attachment comprises fabric.

6. A healing shoe according to claim 1, wherein the decorative attachment comprises paint.

7. A healing shoe according to claim 1, wherein the decorative attachment comprises beads.

8. A healing shoe according to claim 1, wherein the decorative attachment comprises at least one of fabric, paint, beads or costume jewelry.

9. A healing shoe comprising:
a shoe sole;
a shoe upper attached to the shoe sole and including at least one portion that folds over a wearer's foot;
a plurality of interchangeable straps selectively attachable to and detachable from the at least one portion; and
at least one decorative attachment secured to each of the straps, the decorative attachment being different on each of the straps such that each strap comprises a unique appearance.

10. A healing shoe according to claim 9, comprising two portions that fold over the wearer's foot, wherein each of the plurality of straps is selectively attachable to the two portions to secure the shoe on the wearer's foot.

11. A strap attachable to a shoe including attachment structure, the strap comprising:
a shape of material including at least one decorative attachment; and
corresponding attachment structure affixed to the shape of material, the corresponding attachment structure being selectively attachable and detachable to the shoe attachment structure.
